(19) United States
(12) Patent Application Publication
(10) Pub. No.: US 2024/0131309 A1
(43) Pub. Date: Apr. 25, 2024

(54) DILATION DEVICES AND RELATED METHODS

(71) Applicant: Mayo Foundation for Medical Education and Research, Rochester (MN)

(72) Inventors: Elizabeth Rajan, Rochester, MN (US); Jad P. AbiMansour, Rochester, MN (US)

(21) Appl. No.: 18/278,371

(22) PCT Filed: Feb. 23, 2022
(86) PCT No.: PCT/US2022/017447

§ 371 Date: Aug. 21, 2023

Related U.S. Application Data
(60) Provisional application No. 63/153,101, filed on Feb. 24, 2021.

(51) Int. Cl. A61M 29/02; A61B 17/34; A61M 25/10

(52) U.S. Cl. CPC A61M 29/02; A61B 17/3421; A61M 25/10182; A61M 25/10186; A61B 2017/00557; A61B 2017/345

(57) ABSTRACT

A dilator includes a tube including a proximal end, a distal end, and a central lumen therethrough. The proximal end includes a connector configured to connect to an inflation device. The dilator includes a balloon disposed between the proximal end and the distal end. The balloon is configured to transition between a contracted state and an expanded state, wherein the device is insertable into a body passage of a subject in need thereof.

### CROSS-REFERENCE TO RELATED APPLICATIONS

This application claims the benefit of U.S. Provisional Application Ser. No. 63/153,101, filed Feb. 24, 2021. The disclosure of the prior application is considered part of (and is incorporated by reference in) the disclosure of this application.

### TECHNICAL FIELD

This disclosure describes dilation devices and related methods of performing a dilation of a body passage of a subject in need thereof.

### BACKGROUND

A stricture is an abnormal narrowing of a body passage, for example, an esophagus, that can be caused by inflammation or fibrosis. Esophageal strictures and strictures of the gastrointestinal (GI) tract can make it difficult or impossible for food to pass through; thus, often requiring patients to promptly seek treatment, which commonly includes endoscopic dilation. Based on etiology and diameter of the stricture, the patient may be required to undergo frequent endoscopic dilations. For example, some patients require daily self-dilation to maintain the caliber of their esophageal lumen. This leads to multiple patient hospital visits and endoscopic procedures resulting in overall increased expenses and an inconvenience to the patient, not to mention the potential risk of side effects and complications from sedation. The need for remote therapeutic technologies for patient care, including remote stricture treatments, has been thrust to the forefront. Thus, there remains a need to develop a dilation tool that allows a patient to perform self-dilation of a body passage (e.g., a GI body passage).

### SUMMARY

In general, this disclosure describes dilators and related methods. Such dilation devices can be used for performing a dilation of and/or viewing a portion of a body passage (e.g., a GI body passage or lumen) for the treatment of a stricture in the body passage.

In one aspect, this disclosure is directed to a dilator that includes a tube including a proximal end, a distal end, and a central lumen therethrough, the proximal end comprising a connector configured to connect to an inflation device, and a balloon disposed between the proximal end and the distal end, the balloon configured to transition between a contracted state and an expanded state, wherein the device is insertable into a body passage of a subject in need thereof.

Embodiments may include one or more of the following features.

In some embodiments, the body passage is a gastrointestinal passage.

In certain embodiments, the gastrointestinal passage is an esophagus, pylorus, duodenum, or any combination thereof.

In some embodiments, the balloon has a pressure between about 3 atmospheres to about 30 atmospheres when in the inflated configuration.

In certain embodiments, the dilator further includes the inflation device.

In some embodiments, the inflation device is a pump.

In certain embodiments, the inflation device is a syringe.

In some embodiments, the inflation device comprises a pressure gauge.

In certain embodiments, the inflation device comprises a relief valve configured to prevent over-pressurization.

In some embodiments, the distal end comprises a capsule-shaped portion sized for insertion in the body passage.

In certain embodiments, the capsule-shaped portion is removably attached to the distal end.

In some embodiments, the capsule-shaped portion is integrally formed with the distal end.

In certain embodiments, the capsule-shaped portion has an outer diameter of about 1 mm to about 15 mm.

In some embodiments, the capsule-shaped portion has an outer diameter of about 3 mm.

In certain embodiments, the dilator further includes a camera secured to the distal end of the tube.

In some embodiments, the camera is capsule-shaped.

In certain embodiments, the camera includes one or more lenses.

In some embodiments, the one or more lenses each face a different direction.

In certain embodiments, the camera includes one or more internal electronic components configured to process or manipulate data acquired by the camera and one or more illumination sources, and wherein the camera is removably attached to the dilator.

In some embodiments, the balloon is in fluid communication with the central lumen of the tube.

In certain embodiments, a portion of the tube defines one or more openings.

In some embodiments, the balloon is disposed around the one or more openings.

In another aspect, this disclosure is directed to a method of using a dilator for performing a dilation of a body passage in a subject in need thereof. The method includes inserting a tube of the dilator into the body passage of the subject, the tube comprising a proximal end, a distal end, and a central lumen therethrough; and inflating a balloon of the dilator using an inflation device connected to a connector attached at the proximal end of the tube, thereby dilating the body passage of the subject.

Embodiments may include one or more of the following features.

In certain embodiments, the balloon is in fluid communication with the central lumen of the tube.

In some embodiments, the body passage is a gastrointestinal passage.

In certain embodiments, the gastrointestinal passage is an esophagus, pylorus, duodenum, or any combination thereof.

In some embodiments, the method further includes actuating a relief valve of the inflation device to prevent over-pressurization of the balloon.

In certain embodiments, the method further includes acquiring an image of the body passage using a camera secured to the distal end of the tube.

In some embodiments, the method further includes wirelessly transmitting the acquired image of the body passage to a remote receiver using one or more wireless transmitters of the camera.

In certain embodiments, the step of inflating the balloon comprises transitioning the balloon from a contracted state to an expanded state.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Furthermore, the use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Some embodiments of the devices and methods described herein may provide one or more of the following advantages.

First, some embodiments described herein may allow a user (e.g., a patient) to perform self-dilation of narrow (e.g., less than about 18 millimeters (mm)) strictures of a body passage. Dilation of narrow strictures are often required to be performed at a hospital or clinic. Thus, the dilation devices and methods of the disclosure may reduce the number of hospital visits and endoscopic procedures (performed by a clinician) that a patient undergoes. This may result in a reduction of overall medical expenses. Furthermore, this may reduce and/or prevent the risk of exposure to pathogens (e.g., severe acute respiratory syndrome coronavirus 2 (SARS-CoV-2)) or other nosocomial infectious pathogens.

Second, some embodiments described herein may allow a user (e.g., a patient) to perform a self-dilation without the need of sedation. For example, routine endoscopic procedures often require the administration of sedative drugs to a patient in order to minimize anxiety and discomfort. However, the dilation devices described herein can be used without administering a sedative drug. Thus, the dilation devices and related methods of the disclosure may prevent any potential risk of side effects (e.g., inability to drive after the endoscopic procedure) and/or complications caused by drug-induced sedation.

Third, some embodiments described herein may allow a user (e.g., a patient) to perform self-dilation to as a recurrent therapy for strictures (e.g., GI strictures). For example, the user may progressively increase the diameter of a stricture and/or maintain a lumen diameter of an affected body passage.

Fourth, some embodiments described herein may allow a user (e.g., a patient) to perform self-dilation from the comfort of their own home. Thus, this may improve the efficiency of treatment that the patient receives. For example, the patient can avoid traveling to obtain treatment.

Fifth, some embodiments described herein may allow a clinician to remotely visualize images and/or video in real time of the lumen of the body passage during the self-dilation procedure. In other words, some embodiments described herein may facilitate and/or support long-distance clinical health care (e.g., telemedicine). For example, the dilation devices described herein can wirelessly transmit a signal (e.g., one or more of an image and/or a video) in real time to a receiver outside of the body of the patient while the dilation device is within the body passage of the patient in order to facilitate long-distance patient and clinician contact, care, intervention, and/or monitoring.

Unless otherwise defined, all technical and scientific terms used herein have the same meaning as commonly understood by one of ordinary skill in the art to which this disclosure pertains. Although methods and materials similar or equivalent to those described herein can be used to practice the various embodiments, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the disclosure are set forth in the accompanying drawings and the description below. Other features, objects, and advantages of the methods and devices described will be apparent from the description, the drawings, and the claims.

### DESCRIPTION OF DRAWINGS

FIG. 1 is a perspective view of an exemplary dilator.

FIG. 2 is a top view of an exemplary dilator.

FIG. 3 is a top view of the exemplary dilator of FIG. 2 in an extended orientation.

FIG. 4 is a side view of the exemplary dilator of FIG. 2 including an inflation device and a duct.

FIG. 5 is a front view of an automatic injector.

FIG. 6 shows an exemplary workflow using the dilator devices disclosed herein.

FIG. 7 shows images of strictures and dilations of a GI tract in a porcine model acquired with the dilator devices disclosed herein.

Like reference numbers represent corresponding parts throughout.

### DETAILED DESCRIPTION

This document relates to devices and methods for the medical treatment of conditions strictures of body passages. In some embodiments, the body passage is a GI passage. In some embodiments, the GI passage is an esophagus, a pylorus, a duodenum, or any combination thereof. For example, this document provides methods and devices for self-dilation of esophageal, pyloric, and/or duodenum strictures.

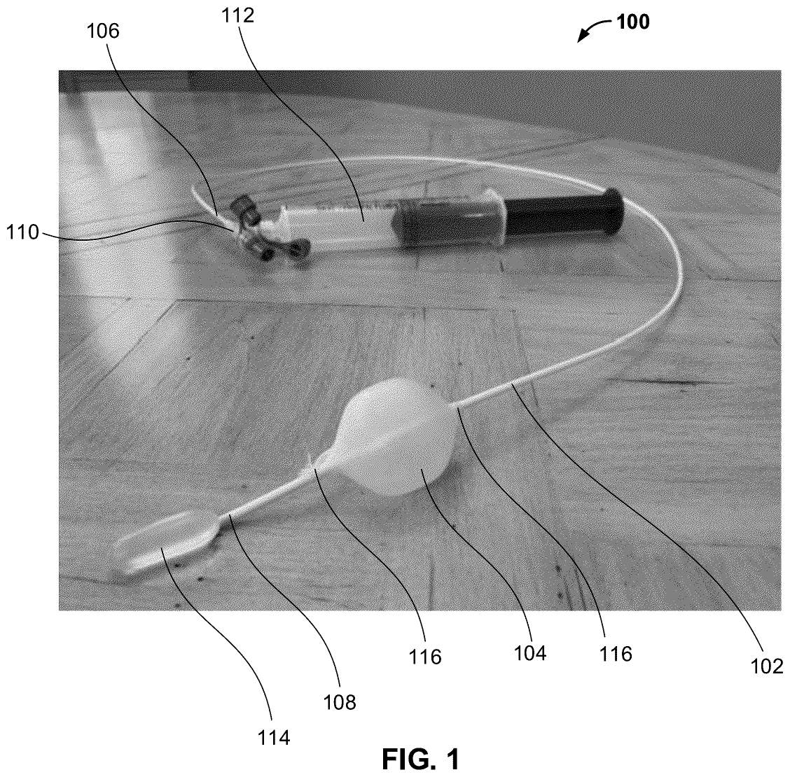

FIG. 1 illustrates a dilator 100 (e.g., a balloon dilator) that can be used to treat a stricture of a subject in need thereof (e.g., a patient). The dilator 100 includes a tube 102 that is formed to be inserted into a body passage (e.g., through the mouth and nose into the esophagus) and a balloon 104 disposed between a proximal end 106 and a distal end 108 of the tube 102.

The tube 102 is an elongate, generally tubular member that is sized to pass through a mouth or nose into an esophagus and further into the remainder of the GI tract, if needed. The tube 102 typically has a total length (e.g., extending between the proximal end 106 and the distal end 108) of about 20.0 centimeters (cm) to about 180.0 cm (e.g., about 70.0 cm). In some embodiments, the tube 102 has a total length of about 20.0 cm to about 30.0 cm, about 30.0 cm to about 40.0 cm, about 40.0 cm to about 50.0 cm, about 50.0 cm to about 60.0 cm, about 60.0 cm to about 70.0 cm, about 70.0 cm to 80.0 cm or more. The tube 102 includes an outer diameter and an inner diameter. The tube 102 typically has an outer diameter of about 0.5 millimeters (mm) to about 5.0 mm (e.g., about 3.0 mm). In some embodiments, the tube 102 has an outer diameter of about 0.5 mm to about 1.0 mm, about 1.0 mm to about 1.5 mm, about 1.5 mm to about 2.0 mm, about 2.0 mm to about 2.5 mm, about 2.5 mm to about 3.0 mm, about 3.5 mm to about 4.0 mm, about 4.5 mm to about 5.0 mm, or more. The tube 102 typically has an inner diameter of about 0.25 mm to about 4.5 mm (e.g., about 2.0 mm). In some embodiments, the tube 102 has an inner diameter of about 0.10 mm to about 0.50 mm, about 0.50 mm to about 0.75 mm, about 0.75 mm to about 1.0 mm, about 1.0 mm to about 1.25 mm, about 1.25 mm to about 1.50 mm, about 1.50 mm to about 1.75 mm, about 1.75 mm to about 2.0 mm, about 2.0 mm to about 2.25 mm, about 2.25 mm to about 2.50 mm, about 2.50 mm to about 2.75 mm, about 2.75 mm to about 3.0 mm, about 3.0 mm to about 3.25 mm, about 3.25 mm to about 3.50 mm, about 3.50 mm to about 3.75 mm, about 3.75 mm to about 4.0 mm, about 4.0 mm to about 4.25 mm, about 4.25 mm to about 4.50 mm, about 4.50 mm to about 4.75 mm, about 4.75 mm to about 5.0 mm, or more. In some embodiments, the tube 102 has an inner diameter of about 2.0 mm.

The tube 102 is a flexible tube typically made of one or more materials that are flexible enough to allow the tube 102 to bend to be appropriately placed within a body passage of a patient as desired and by natural peristalsis or motility of the gut. Example materials from which the tube 102 is typically made include nylon, polysulfone, silicone, vinyl, polyurethane and polyether ether ketone (PEEK). The tube 102 is typically manufactured primarily via extrusion and via secondary processes that may include one or more of punching, laser cutting, forming, and/or printing. The tube 102 can further include ruled markings (omitted from the figures) on an external surface of the tube 102 that indicate a distance of the tube 102 from the incisor teeth of the patient to a distal end 108 of the tube 102. In some embodiments, the ruled markings indicate distances from the proximal end 106 or from the distal end 108 of the tube 102. The ruled markings can be viewed by a user during a dilation procedure to determine a depth to which the tube 102 has been inserted into the patient. The ruled markings may be provided in metric units or English units or provided as a dimensionless scale. In some embodiments, the ruled markings are evenly distanced from one another (e.g., distanced at every 10 cm).

The tube 102 has a proximal end 106, a distal end 108, and a central lumen defined therethrough. The proximal end 106 of the tube 102 includes a connector 110 configured to connect to an inflation device 112 such that the tube 102 and the inflation device 112 are in fluid connection with each other. In some examples, the inflation device 112 is a syringe, as shown in FIG. 1. In some embodiments, the dilator 100 includes the inflation device 112. In some embodiments, the inflation device 112 is a pump. The inflation device 112 can be fluidically connected to a pressure gauge that indicates the pressure of a fluid (e.g., gas) that is being delivered through the tube 102. As such, the pressure gauge can help a user achieve a desired pressure. In some embodiments, the inflation device 112 and the pressure gauge may be incorporated as a single device attached to the proximal end 106 of tube 102. In some examples, the inflation device and the pressure gauge may have a pre-determined maximum pressure allowable. In some embodiments, the inflation device may be automated. For example, the inflation device can include a button that can be actuated (e.g., pressed) to automatically dilate the balloon to a preset pressure. The inflation device 112 can further include a relief valve configured to prevent over-pressurization of the dilator 100. For example, when a desired pressure in the dilator 100 is reached, the relief valve can automatically release excess fluid, thereby preventing excessive pressure build-up in the dilator 100. In some embodiments, the inflation of the balloon may occur without the connector 110. For example, the inflation of the balloon may occur wirelessly.

A portion of the tube 102 further defines one or more openings (e.g., sidewall openings) through which a fluid can be delivered. For example, the central lumen allows fluid (e.g., a gas) to flow from the inflation device 112, through the central lumen, to one or more openings on the portion of the tube 102. In some embodiments, the portion of the tube 102 defining one or more openings is closer to the distal end 108 of the tube than to the proximal end 106 of the tube 102. In some embodiments, the portion of the tube 102 defining one or more openings is about 1 cm to about 20 cm or more above the distal end 108 of the tube 102. In some embodiments, one or more openings defined by a portion of the tube 102 are at or near the distal end 108 of the tube 102. In some embodiments, the dilator 100 further includes an inflation port or an inflation lumen in the tube 102. In some embodiments, the inflation port or the inflation lumen is in fluid communication with the central lumen of the tube 102, the balloon 104, and the inflation device 112, thereby facilitating the inflation of balloon 104. In some embodiments, the balloon may be inflated via a separate tube that may be attached inside or outside the tube 102.

The balloon 104 is disposed around one or more openings defined by a portion of the tube 102, such that the balloon 104 creates two opposing seals 116 on the portion of the tube that defines one or more openings. In some embodiments, the inflation of the balloon 104 may be achieved through one or more openings defined by a portion of the tube 102 and/or via an inflating port/lumen that is built internally into the tube 102. The balloon may be an integral part of the tube 102. For example, in some embodiments, one or more portions of the balloon (e.g., opposing end portions) are integrally formed with the tube 102. In some embodiments, the balloon 104 may be attached to the tube 102 (e.g., via sutures, glue, bands, or similar mechanisms) to secure it in position. The balloon 104 is in fluid communication with the central lumen of the tube 102. The balloon 104 is configured to transition between a contracted state (e.g., deflated state) and an expanded state (e.g., inflated state). The seals 116 allow a user to deliver a fluid from the inflation device 112, through the central lumen of the tube 102, through one or more openings defined by a portion of the tube 102, and into the balloon 104. The seals 116 permit the fluid (e.g., a pressurized fluid) being delivered to the balloon 104 to fill and be retained by an interior volume of the balloon 104, thereby inflating the balloon 104 to an expanded state. In some embodiments, the dilator 100 includes one or more balloons.

The balloon 104 is a compliant balloon typically made of one or more materials that are flexible enough to allow the balloon 104 to be inflated to an expanded volume that is greater than a contracted volume of the balloon 104 in a contracted state. Example materials from which the balloon 104 is typically made include nylon, polyether block amide, silicone, polyethylene, latex, or polyethylene terephthalate (PET). In some cases, the balloon 104 can be inflated with a liquid or a gas. The balloon has a substantially spherical shape when in the expanded state. In some embodiments, the balloon has a substantially oval, elongated, tubular, or any other suitable shape when in the expanded state. In some cases, the balloon 104 can typically be inflated to a diameter of about 5 mm to about 20 mm (e.g., about 5 mm to about 10 mm, about 5 mm to about 15 mm, about 10 mm to about 15 mm, about 12 mm to about 15 mm, or about 14 mm to about 20 mm). In some embodiments, the balloon 104 is inflated to a diameter of about 14 mm. In some embodiments, the balloon 104 is inflated to a diameter of about 5 mm. In some embodiments, the balloon 104 has a length ranging from about 40 mm to about 120 mm (e.g., about 40 mm to about 50 mm, about 40 mm to about 80 mm, about 50 mm to about 60 mm, about 40 mm to about 60 mm, about 40 mm to about 70 mm, about 50 mm to about 80 mm, about 60 mm to about 80 mm, about 70 mm to about 80 mm, about 80 mm to about 90 mm, about 80 mm to about 100 mm, about 80 mm to about 110 mm, or about 80 mm to about 120 mm). In some embodiments, the balloon 104 has a length of about 80 mm. In some embodiments, the balloon 104 has a length of about 50 mm. In some embodiments, the balloon 104 in an expanded state has a diameter that is 2, 3, 4, 5, 6, 7, 8, 9, or 10 times larger than the diameter of the balloon 104 in a contracted state. The balloon 104 can have a pressure ranging from about 3 atmospheres to about 30 atmospheres when in the expanded state. In some embodiments, the balloon 104 can have a pressure ranging from about 1 atmospheres to about 5 atmospheres, about 5 atmospheres to about 10 atmospheres, about 10 atmospheres to about 15 atmospheres, about 15 atmospheres to about 20 atmospheres, about 20 atmospheres to about 25 atmospheres, about 25 atmospheres to about 30 atmospheres, about 30 atmospheres to about 35 atmospheres, about 35 atmospheres to about 40 atmospheres, or more when in the expanded state. The balloon 104 typically has a length of about 1 cm to about 10 cm. In some embodiments, the balloon 104 is a hydrostatic balloon.

The distal end of tube 102 includes a capsule-shaped portion 114 sized for insertion in the body passage. In some embodiments, the capsule-shaped portion 114 is removably attached to the distal end 108. For example, the capsule-shaped portion 114 may be attached to the distal end 108 via a string, a snap-fit connector, a press-fit connector, or a similar attachment mechanism. In some embodiments, the capsule-shaped portion 114 is integrally formed with the distal end. The capsule-shaped portion 114 typically has an outer diameter of about 1 mm to about 15 mm (e.g., about 1 mm to about 3 mm, about 1 mm to about 5 mm, about 1 mm to about 10 mm, about 1 mm to about 15 mm, about 3 mm to about 5 mm, about 3 mm to about 10 mm, about 3 mm to about 15 mm, about 5 mm to about 10 mm, about 5 mm to about 15 mm, or about 10 mm to about 15 mm). In some embodiments, the capsule-shaped portion 114 has an outer diameter of about 3 mm. In some embodiments, the capsule-shaped portion 114 has an outer diameter of about 14 mm. In some embodiments, the capsule-shaped portion is spherocylindrical shape (i.e., a cylinder having hemispherical ends). In some embodiments, the capsule-shaped portion is sized and shaped as a multi-vitamin capsule (e.g., a capsule sized for human consumption). In some embodiments, the capsule-shaped portion may be hollow defining an interior volume that may be filled with a fluid (e.g., a fluid). In some embodiments, the capsule-shaped portion may be solid.

The capsule-shaped portion 114 typically made of one or more materials that are pliable enough to allow the capsule-shaped portion 114 to be easily swallowed by a user (e.g., a patient). Example materials from which the capsule-shaped portion 114 is typically made include silicone, polycarbonate, ethyl cyanoacrylate, vinyl, polyurethane, or polyethylene.

The dilator 100 can further include a camera secured to the distal end 108 of the tube 102. The camera may be secured within a holder or cradle or similar object that is connected (e.g., removably connected or integrally connected) to the distal end 108 of the tube 102. In some embodiments, the camera is removably attached to the dilator 100. In some embodiments, the camera is removably attached to the distal end 108 of the tube 102. In some embodiments, the camera may be located adjacent to a proximal end or a distal end of the balloon 104. In some embodiments, the camera may be located in any suitable location between the distal end 108 and a proximal or distal end of balloon 104. In some embodiments, the camera may be reversibly attached to a portion of the tube 102 between the distal end 108 and a proximal or distal end of balloon 104. In some embodiments, the camera is adjacent to a distal edge of the balloon such that the camera is able to transmit views of the balloon while it is being inflated or deployed. In some embodiments, the camera has a capsule-like shape.

In some embodiments, the camera can have a capsule-shaped body and be sized to be swallowable by a user (e.g., a patient). For example, the camera can have an outer diameter of about 1 mm to about 15 mm (e.g., about 1 mm to about 3 mm, about 1 mm to about 5 mm, about 1 mm to about 10 mm, about 1 mm to about 15 mm, about 3 mm to about 5 mm, about 3 mm to about 10 mm, about 3 mm to about 15 mm, about 5 mm to about 10 mm, about 5 mm to about 15 mm, or about 10 mm to about 15 mm). In some embodiments, the camera has an outer diameter of about 3 mm. In some embodiments, the camera includes one or more lenses that are configured to image an interior of a body passage. In some embodiments, each of the lenses face a different direction such that the camera can provide a view in one or more different angles (e.g., a 360 degree view). For example, the camera can have one or more lenses on a side of the body of the camera or on a distal and/or proximal end of the body of the camera. The camera can include one or more internal electronic components configured to process or manipulate data acquired by the camera. In some embodiments, the camera is configured to record and/or transmit data in real time. In some embodiments, the data is an image, a video, a sound, or any combination thereof.

The camera may include an imager for capturing images and/or video and an illumination source for illuminating the body passage. In some embodiments, the illumination source (e.g., a light emitting diode (LED) light) can be powered wirelessly (e.g., via one or more batteries). In some embodiments, the illumination source is configured to help visualize an interior of a body passage (e.g., the GI tract). In some embodiments, the illumination source has adjustable brightness levels. For example, in some embodiments, a user can adjust the brightness levels when the camera is outside and inside a body passage of a patient. In some embodiments, the data can be viewed remotely. For example, in some embodiments, the data can be viewed in a mobile computing device (e.g., a smart phone, a laptop, a tablet, or the like). In some embodiments, the data can be viewed in a computing device (e.g., a network computing device, a personal computer, or the like).

In some embodiments, the camera lens is protected by a cover (e.g., a clear plastic dome) configured to prevent smudging and/or blurring of the lens by body passage contents (e.g., lumen contents) when inserted in a body passage. In some embodiments, the camera is configured to transmit views that are forward, retrograde, and/or 360 degrees. In some embodiments, the camera is an antegrade and retrograde camera (e.g. a dual camera).

In some embodiments, the electronic components include electronic circuitry and a transmitter to control the camera functionalities such as transmitting data to a data receiver. In some embodiments, the electronic components include a wireless transmitter configured to wirelessly transmit acquired data to a receiver that is not physically connected (e.g., via wires) to the wireless transmitter and is external to the body of the user (e.g. a patient). In some embodiments, the camera uses RF sources, wireless networks (e.g., Wi-Fi), and/or cellular communications to wirelessly transmit data to the data receiver. The electronic circuitry and the transmitter may include, for example, an input-output (“I/O”) interface/device, one or more controllers and a camera receiver. The camera receiver may be used, for example, to receive control information (e.g., to change a mode of operation, to change the value of a parameter, etc.). The camera may include an optical system, including, for example, one or more lenses and/or mirrors that may be used to focus reflected light onto the imager.

The dilator 100 can further include a data receiver, preferably including a processor, which may receive data from the camera. The processor may be, for example, a digital signal processor or any other real time processor or controller. In some embodiments, the data receiver may include a storage unit for storing the received data. Alternatively, in other embodiments, the data may not be stored in the receiver and may either be transmitted or transferred to another storage unit or may not be stored at all.

A dilator may be substantially similar in construction and function in several aspects to the dilator 100 discussed above, but can include: 1) an automatic injector 400, 2) an imaging system including a video transmitter 220 configured to connect with the tube of the dilator 200, and 3) a duct 224. In some embodiments, the automatic injector 400 is configured to automatically inflate and deflate the balloon of the dilator. In some embodiments, the video transmitter 220 is a wireless video transmitter. In some embodiments, the video transmitter 220 can be configured to connect to a wireless network (e.g., Wi-Fi) to enable live or real-time viewing and/or recording of video output from the camera 218. In some embodiments, the duct 224 is a wire duct.

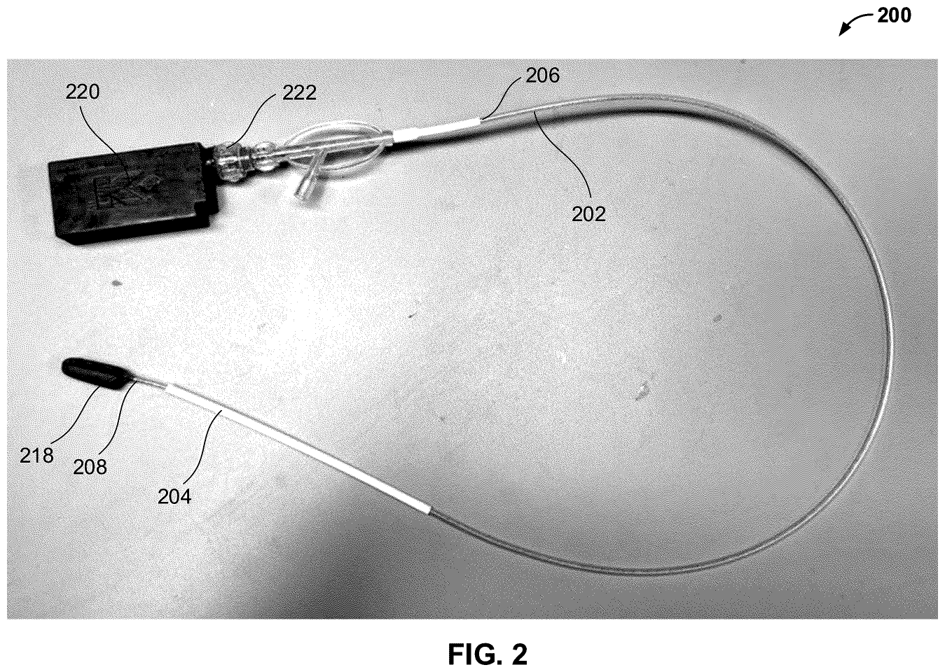

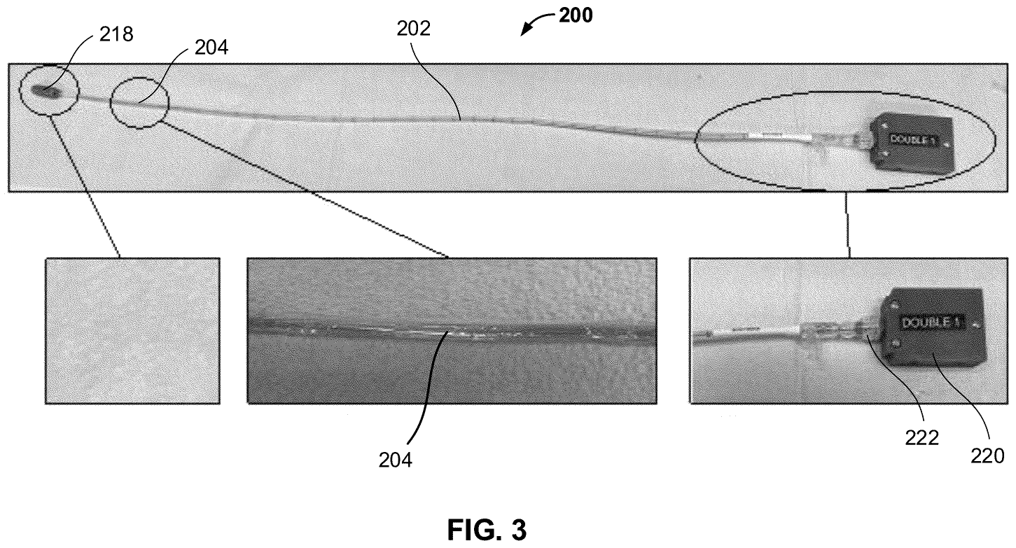

FIGS. 2, 3, and 4 illustrate examples of a dilator 200 (e.g., a balloon dilator) that can be used to treat a stricture of a subject in need thereof (e.g., a patient). The dilator 200 includes a tube 202 that is formed to be inserted into a body passage (e.g., through the mouth and nose into the esophagus) and a balloon 204 disposed between a proximal end 206 and a distal end 208 of the tube 202. The imaging system of the dilator 200 is substantially similar in construction and function to the imaging system of the dilator 100 and, accordingly, includes the camera 218, one or more of the illumination sources (e.g., LEDs), and the electronic components such as electronic circuitry and a transmitter to control the camera functionalities, except that the transmitter can be a video transmitter 220 configured to connect to a tube 202 of the dilator 200. In some embodiments, the video transmitter 220 can include an adaptor 222 configured to connect to a syringe. In some embodiments, the adaptor 222 is a slip tip connector or a locking connector.

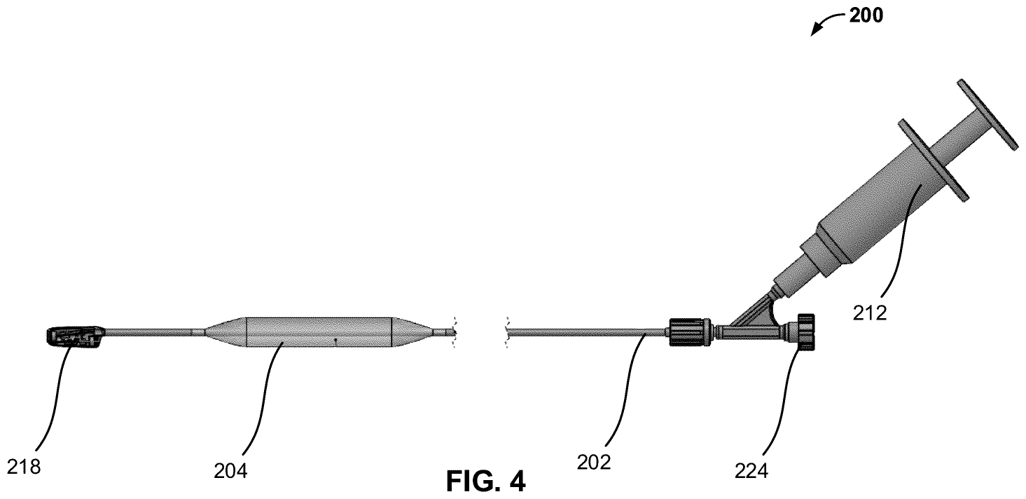

FIG. 4 shows an example of a dilator 200 including the inflation device 212 and a duct 224. In some embodiments, duct 224 is a tube, opening, or passageway configured to house one or more wires (e.g., imaging system wires). In some embodiments, the duct 224 is a wire bundle exit. In some embodiments, the duct 224 is in fluid communication with the tube 202. In some embodiments, the duct 224 is positioned adjacent to a proximal end of the tube 202. In some embodiments, the duct 224 is positioned adjacent to a connection of the tube 202 with the inflation device 212.

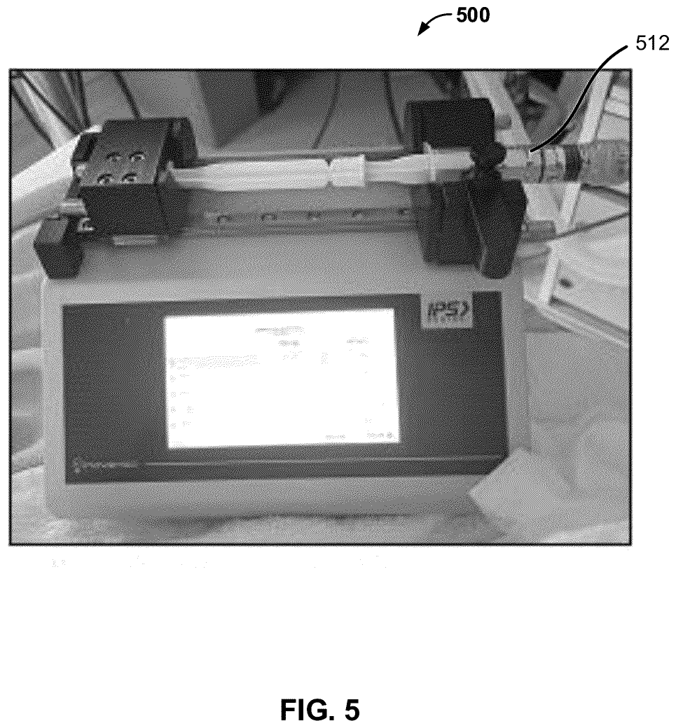

FIG. 5 shows an exemplary automatic injector 500 configured to operatively connect with the inflation device 512. In some embodiments, the automatic injector 500 is in fluid communication with the tube and balloon of the dilator. In some embodiments, the automatic injector 500 can be configured to automatically inflate and/or deflate the balloon of the dilator. In some embodiments, the automatic injector 500 can be configured to reach a pre-set volume and a pre-set pressure. In some embodiments, the automatic injector 500 is a syringe pump. Disclosed herein, in certain embodiments, are methods of using the dilator devices of the disclosure for performing a dilation of a body passage in a subject in need thereof. First, once the location (e.g., the distance from the incisor teeth of a patient to a stricture in the patient), etiology, and inner diameter of a stricture (e.g., a GI stricture) has been established using an endoscopic procedure (e.g., an upper endoscopy or deep enteroscopy), the patient is provided the dilation device of the disclosure with a tube 102 having an appropriate size based on the parameters determined by the endoscopic procedure. In some embodiments, the stricture has a diameter of about 5 mm to about 18 mm. The dilation device of the disclosure is provided to the patient for self-dilation (e.g., dilation to be performed by the patient at home). The size of the one or more balloons will be determined based on baseline diameter of the stricture(s) (e.g., GI stricture(s)) of the patient.

The methods can include inserting the tube 102 of the dilator 100 into the body passage of the subject. For example, the user (e.g., a patient) swallows the capsule-shaped portion 114 of the tube 102 while drinking some liquid (e.g., water). Prior to starting the self-dilation procedure, the patient may optionally numb her or his throat as needed (e.g. by using lidocaine throat spray) to reduce any potential discomfort. Once the ruled markings on the tube 102 indicate that the tube 102 and/or balloon 104 are beyond the location of the stricture, the user (e.g., a patient) pulls the tube 102 back out of her or his mouth so that the balloon 104 is aligned with the stricture. For example, in an esophageal stricture case, once the ruled markings on the tube 102 indicate that the tube 102 and/or balloon 104 are beyond the location of the stricture, or have reached the stomach (e.g., a stricture located at a depth of 30 cm and a ruled marking of the tube 102 indicating that a distal end 108 of the tube 102 or the balloon 104 is at a depth of 40 cm), the user pulls back the tube 102 around its proximal end 106 or at the mouth so that the balloon 104 is situated at the level of the stricture.

Next, the balloon 104 is inflated using the inflation device 112, and the balloon 104 transitions from its initial, contracted state to an expanded (e.g., inflated) state. In some embodiments, the balloon 104 is inflated to a pre-determined preferred pressure (e.g., about 3 atmospheres to about 30 atmospheres). In some embodiments, the pressure of the balloon 104 is not pre-determined and instead, is manually controlled by the user. For example, the user may also manually inflate the balloon 104. In such an example, the methods can further include manually or automatically actuating a relief valve of the inflation device 112 to prevent over-pressurization of the balloon 104. The inflation of balloon 104 may be sustained for about 1 minute and may be repeated, as prescribed by the physician. In some embodiments, the inflation is sustained for about 30 seconds to about 3 minutes (e.g., for about 30 seconds to about 1 minute, about 1 minute to about 1.5 minutes, about 1.5 minute to about 2 minutes, about 2 minutes to about 2.5 minutes, about 2.5 minutes to about 3 minutes). In some embodiments, the step of inflating the balloon 104 includes transitioning the balloon 104 from a contracted to an expanded state and back to a contracted state (e.g., an inflation and deflation cycle). In some embodiments, an inflation and deflation cycle of the balloon 104 is repeated once, twice, three, four, five, six, seven, eight, nine, ten or more times as prescribed and/or as needed by the patient. Once the inflation and deflation cycle is completed, the balloon 104 is deflated, and the tube 102 is pulled out through the mouth of the patient (e.g., by the patient herself or himself).

In some embodiments, the methods further include acquiring an image of the body passage using a camera secured to the distal end 108 of the tube 102. In some embodiments, when the dilator 100 includes the camera secured to a distal end 108 of the tube 102, the user (e.g., the patient) can activate the camera prior and/or during the self-dilation procedure to begin acquiring, recording, and/or transmitting data. In some embodiments, the method further includes wirelessly transmitting the acquired image of the body passage to a remote receiver using one or more wireless transmitters of the camera. In some embodiments, the physician may remotely visualize the recorded and/or transmitted data and provide the patient with long-distance patient-physician intervention and/or monitoring. In some embodiments, the recorded and/or transmitted data includes dilation and/or post-dilation images that may include mucosal changes on an interior lumen of the body passage of the patient. In some embodiments, the physician remotely visualizes the recorded and/or wirelessly transmitted data in real time as the patient performs one or more of the inflation and deflation cycles. In some embodiments, the physician instructs the patient when to start and/or stop the one or more inflation and deflation cycles. Once the inflation and deflation cycle(s) are concluded, the tube 102 is pulled out through the mouth of the patient.

In some embodiments, the dilator 100 or any of its components may be single-use or reusable. For example, the dilator 100 may be provided to the user as a sterile single-use kit that is to be disposed of after the self-dilation procedure is completed. Alternatively, in some embodiments, the entire or parts of the dilator 100 may be reusable and amenable to be sterilized or disinfected (e.g., via autoclave sterilization) in between uses.

### EXAMPLES

Certain embodiments of the present disclosure are further described in the following examples, which do not limit the scope of any embodiments described in the claims.

### Example 1—Exemplary Workflow Using a Dilator Device in a Porcine Model

The dilator device disclosed herein was tested in a porcine model. Briefly, an esophageal stricture was induced in all pigs via endoscopic mucosal resection at day 0. At day 14, the upper endoscopy was repeated to assess the stricture. The stricture was dilated using the dilator device described herein and the dilation was assessed endoscopically. Lastly, the pigs were sacrificed and necropsies were performed to assess the GI tracts.

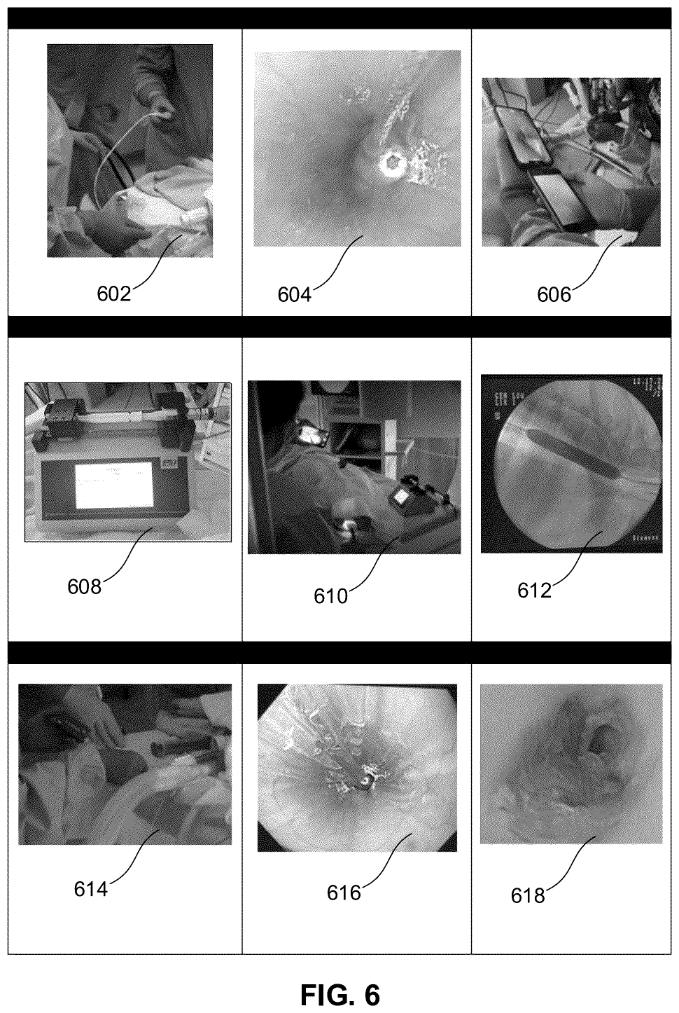

FIG. 6 shows an overview of an exemplary workflow using the dilator devices of the disclosure in a porcine model. Steps 602, 604, and 606 describe how the dilator device was advanced through an overtube into an esophagus and past a stricture in a GI tract. Step 602 illustrates how the dilator device was inserted through an overtube. Step 604 illustrates the endoscopic-assisted placement of the dilator device in a porcine model due to lack of peristalsis. Step 606 illustrates the wireless video transmission of video output from the camera via Wi-Fi. Steps 608, 610, and 612 describe how the balloon of the dilator device was automatically inflated using an automatic injector. Step 608 illustrates how the automatic injector was in fluid connection with a tube of the dilator and inflated the balloon. In some embodiments, manual inflation of the balloon of the dilator device can also be performed. In some embodiments, manual inflation of the balloon can be performed by injecting about 15 to about 16 cubic centimeters (cc) of a liquid (e.g., saline). Step 612 illustrates a fluoroscopic view of the balloon in an expanded state. Steps 614, 616, and 618 describe the withdrawal of the deflated balloon and inspection of the mucosa. The catheter was withdrawn while monitoring the Wi-Fi video feed (step 614). Next, the catheter withdrawal was endoscopically visualized, and a retrograde-facing camera can be seen in the distal esophagus (step 616). An example of mucosal tear after the successful dilation was performed is shown in step 618.

### Example 2—Assessment of Induced Strictures and Dilations in a Porcine Model

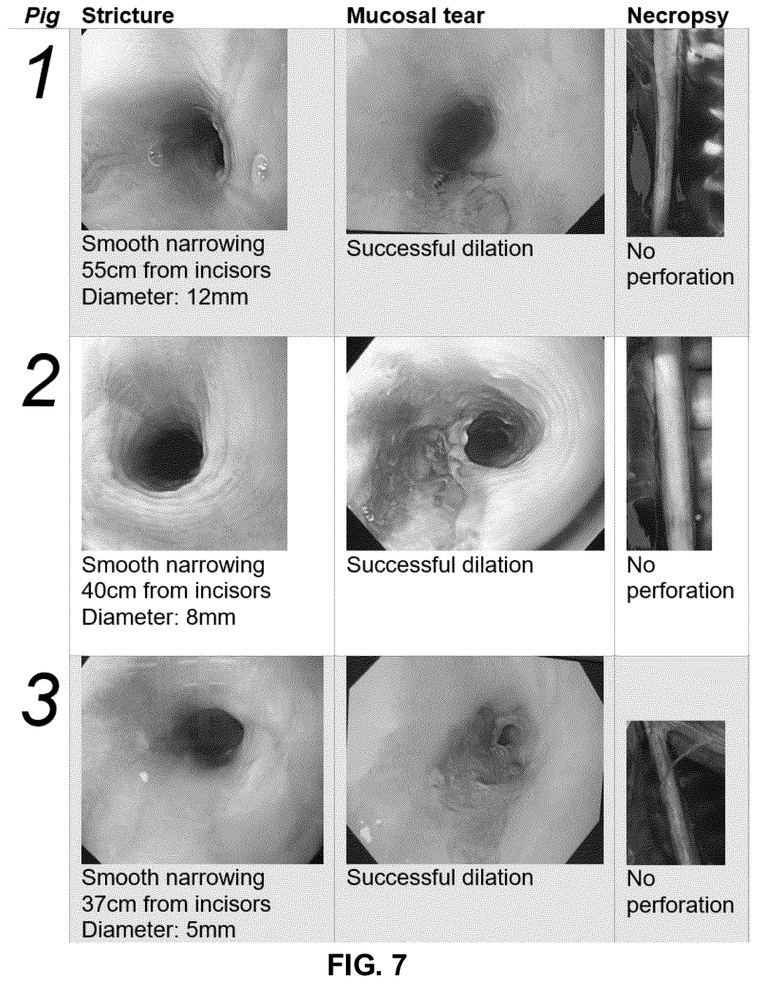

FIG. 7 is a group of images showing the induced strictures and successful dilations performed in a porcine model (n=3) using the dilator devices of the disclosure. The induced strictures had diameters of about 12 mm (pig #1), 8 mm (pig #2), and 5 mm (pig #3). All strictures exhibited smooth narrowing and were located at about 55 cm from incisors (pig #1), at about 40 cm from incisors (pig #2), and at about 37 cm from incisors (pig #3). Images of the mucosal tears show successful dilations were performed with the dilator device with no observed perforations.

### Other Embodiments

While this specification contains many specific implementation details, these should not be construed as limitations on the scope of any method or device or of what may be claimed, but rather as descriptions of features that may be specific to particular embodiments of particular methods and devices. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described herein as acting in certain combinations and even initially claimed as such, one or more features from a claimed combination can in some cases be excised from the combination, and the claimed combination may be directed to a sub-combination or variation of a sub-combination.

Particular embodiments of the subject matter have been described. Other embodiments are within the scope of the following claims. For example, the actions recited in the claims can be performed in a different order and still achieve desirable results.

1.-30. (canceled)

31. A dilator comprising:
a tube comprising a proximal end, a distal end, and a central lumen therethrough, the proximal end comprising a connector configured to connect to an inflation device; and
a balloon disposed between the proximal end and the distal end of the tube, the balloon configured to transition between a contracted state and an expanded state,
wherein the dilator is insertable into a body passage of a subject in need thereof, and
wherein the distal end of the tube comprises a capsule-shaped portion that is sized for insertion in the body passage.

32. The dilator of claim 31, wherein the body passage is a gastrointestinal passage, and wherein the gastrointestinal passage is an esophagus, pylorus, duodenum, or any combination thereof.

33. The dilator of claim 31, wherein the balloon has a pressure between about 3 atmospheres to about 30 atmospheres when in an expanded configuration.

34. The dilator of claim 31, further comprising the inflation device, wherein the inflation device is a pump or a syringe.

35. The dilator of claim 31, wherein the inflation device comprises a pressure gauge.

36. The dilator of claim 31, wherein the inflation device comprises a relief valve configured to prevent over-pressurization.

37. The dilator of claim 31, wherein the capsule-shaped portion is removably attached to the distal end of the tube.

38. The dilator of claim 31, wherein the capsule-shaped portion is integrally formed with the distal end of the tube.

39. The dilator of claim 31, wherein the capsule-shaped portion has an outer diameter of about 1 mm to about 15 mm.

40. The dilator of claim 31, wherein the capsule-shaped portion has an outer diameter of about 3 mm.

41. The dilator of claim 31, further comprising a camera secured to the distal end of the tube.

42. The dilator of claim 41, wherein the camera is capsule-shaped.

43. The dilator of claim 42, wherein the camera comprises one or more lenses.

44. The dilator of claim 43, wherein the one or more lenses each face a different direction.

45. The dilator of claim 44, wherein the camera comprises one or more internal electronic components configured to process or manipulate data acquired by the camera and one or more illumination sources, and wherein the camera is removably attached to the dilator.

46. The dilator of claim 31, wherein the balloon is in fluid communication with the central lumen of the tube.

47. The dilator of claim 31, wherein a portion of the tube defines one or more openings, and wherein the balloon is disposed around the one or more openings.

48. A method of using a dilator for performing a dilation of a body passage in a subject in need thereof, the method comprising:
inserting a tube of the dilator into the body passage of the subject, the tube comprising a proximal end, a distal end, and a central lumen therethrough;
inflating a balloon of the dilator using an inflation device connected to a connector attached at the proximal end of the tube, thereby dilating the body passage of the subject; and
actuating a relief valve of the inflation device to prevent over-pressurization of the balloon.

49. The method of claim 48, wherein the balloon is in fluid communication with the central lumen of the tube, wherein the body passage is a gastrointestinal passage, and wherein the gastrointestinal passage is an esophagus, pylorus, duodenum, or any combination thereof.

50. The method of claim 48, further comprising acquiring an image of the body passage using a camera secured to the distal end of the tube, and wirelessly transmitting the acquired image of the body passage to a remote receiver using one or more wireless transmitters of the camera.
